(19) United States
(12) Patent Application Publication
(10) Pub. No.: US 2010/0292727 A1
(43) Pub. Date: Nov. 18, 2010

(54) FACE WRINKLE CORRECTION AND STRESS RELIEF DEVICE

(76) Inventor: ELIZABETH BLOCH, HAVERTOWN, PA (US)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(21) Appl. No.: 12/779,508

(22) Filed: May 13, 2010

Related U.S. Application Data
(60) Provisional application No. 61/177,760, filed on May 13, 2009.

(51) Int. Cl. A61F 5/00

(52) U.S. Cl. 606/204.35

(57) ABSTRACT

An article and method of reducing and correcting facial wrinkles on a person when the person is resting one of its faces on a surface, such as a pillow. The article is secured to the person's head substantially above the face to be protected. The article has a cushioned section having a thickness sufficient to elevate the face a sufficient distance to reduce the pressure on the face from the flat surface when resting thereon.

### CROSS REFERENCE TO RELATED APPLICATIONS

The present application claims priority to U.S. Provisional Patent Application Ser. No. 61/177,760 filed May 13, 2009, the disclosure of which is hereby incorporated by reference herein.

### FIELD OF THE INVENTION

The present invention relates to a head support article for reducing and correcting facial wrinkles that can also be used for stress relief. The present invention also provides a method for doing the same.

### BACKGROUND OF THE INVENTION

As people age, skin has a natural tendency to lose some of its elasticity and smoothness. These effects are largely attributable to environmental considerations, normal wear and tear, and the genetic make-up of the person. With respect to the environment, a myriad of factors including ultraviolet radiation, pollution, wind, heat, humidity, and harsh chemicals all have been shown to be contributing factors. Choices such as smoking, poor nutrition, excess alcohol consumption and stress have also been shown to accelerate the aging process. These all may be compounded by the genetic make-up of the person.

It is also known that physical manipulation of the facial structure over long periods of time may similarly affect aging or contribute to the appearance of aging. For example, when a person sleeps on his or her side or stomach, or tosses and turns while sleeping, the person's face may be pressed into the pillow. This tends to pull, manipulate and wrinkle the skin while sleeping. These effects are often observable in the morning as the skin may have residual wrinkles.

At a younger age, these wrinkles often fade during the course of the morning, though sometimes over a few hours. As a person ages, however, the deepened wrinkles no longer recede. These wrinkles often deepen as one ages, especially between the nose and cheek area. The forehead and chin area also begin to wrinkle giving a more saggy and droopy appearance.

New and established products in the wrinkle-fighting industry are well known, many of which are available as lotions, creams, nutritional supplements and the like. Purchasing these products incurs an ongoing cost to customers who spend tens to hundreds of dollars monthly or even weekly. In addition, the use of these products may be time consuming and detract from a person's valuable daily time, which could be used addressing other priorities.

Other, and much more radical, approaches to fighting aging are also available and include cosmetic surgery, laser treatment, chemical exfoliation, dermabrasion or skin sanding, collagen injections, and Botulinum Toxin Type A injections. These methods are all significantly more expensive. Often, they must be performed repeatedly to maintain the desired appearance, and, in certain instances, pose significant risks to the person up to and including death.

Accordingly, an article and method are desirable for reducing and correcting facial wrinkles that is inexpensive and easy to use. The present invention addresses the foregoing needs.

### SUMMARY OF THE INVENTION

In one embodiment, the present invention relates to an article for reducing facial wrinkles when the person is resting a side of his or her face on a surface. The article includes a longitudinally extending head support mountable on a person's forehead and having first and second ends. The head support is flexible for conforming to the forehead and is of sufficient length between the first and second ends to be capable of extending from a first position on a side of the person's head approximately above the person's ear near the temple area, across the person's forehead, and to a second position approximately above the other ear near the other temple area. The article also includes a means for securing the article to the person's head, such as a strap.

In one embodiment the head support includes a cover and at least one insert receivable within the cover. The cover is made of any suitable material that would be comfortable against the subject's skin.

The cover is formed to include a bottom portion and a top portion, as well as a first and second ends. In certain embodiments, the bottom portion includes one or more bulge elements, which are used to cover the person's eyes or otherwise provide counter pressure at or about the person's eyes. The top portion of the cover may include one or multiple openings for receiving the insert.

The insert may be formed of one or more fabric pieces folded or otherwise configured to provide a cavity there within. Much like the cover, the insert may be made from any suitable material that would be comfortable against the person's skin. The insert is filled with a cushion or resiliently compressible fill material, which is used to support the wearer's head during use.

In further embodiments, a scented aroma may be embedded or otherwise provided with the article to support aroma therapy.

The instant invention also relates to a method of reducing facial wrinkles on a person when the person is resting a side of his or her face on a surface, such as a pillow. More specifically an article is secured to the person's head substantially above the ear on at least a first side of the person's head. The article includes a cushioned section extending from at least a side forehead area above the first face to an area approximately above the ear.

When the person rests the side of his or her head against the surface using the foregoing method, the article elevates and/or angles the temple area of the head so as to reduce the pressure from the surface on the face, particularly the cheek area. This is advantageous because it minimizes physical manipulation of the person's face by the surface and prevents it from causing deepening wrinkles. With repeated use, the instant invention is believed to be capable of minimizing deep set wrinkles that have previously appeared on the face.

Additional embodiments and advantages will be apparent to one of skill in the art based on the disclosure provided herein.

### BRIEF DESCRIPTION OF THE FIGURES

FIG. 1 illustrates a side view perspective of a person wearing the article of the present invention while sleeping;

FIG. 2 illustrates a rear perspective view of the article of the present invention;

FIG. 3 illustrates an exploded view of the article of the present invention; and

FIG. 4 illustrates a cross sectional view of the article of the present invention taken along line 4-4 of FIG. 2.

### DETAILED DESCRIPTION OF THE INVENTION

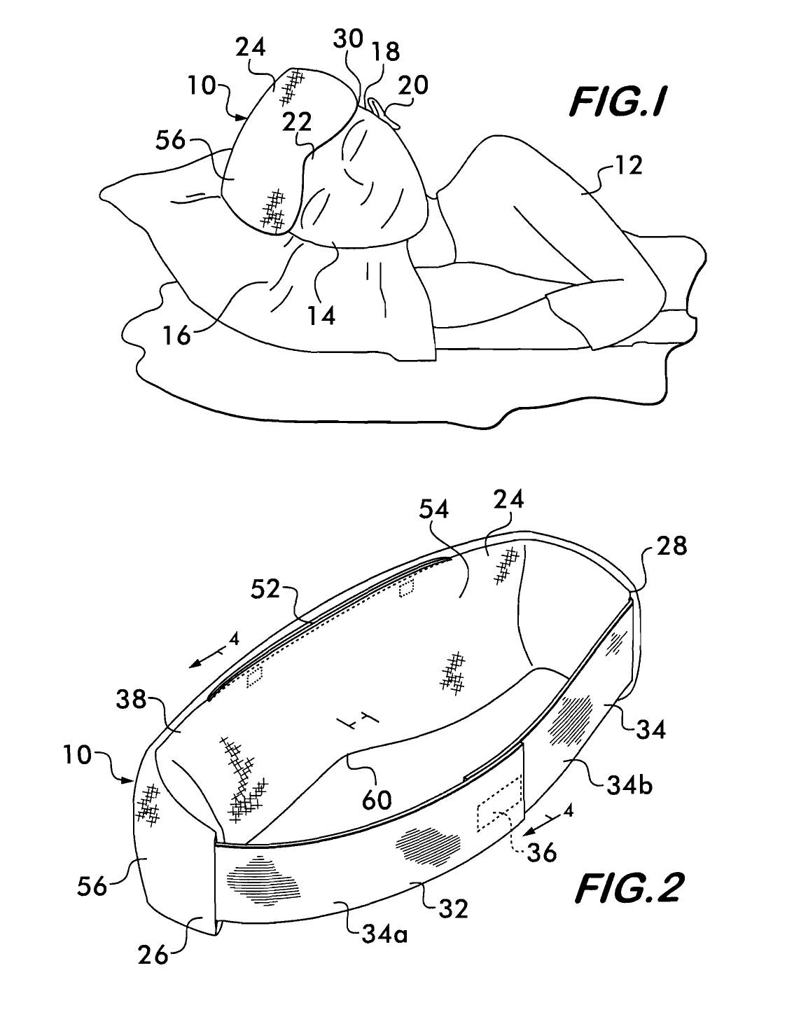

Referring initially to FIGS. 1 and 2, the present invention relates to a removable head support article 10 for reducing and correcting facial wrinkles when a person 12 rests his or her face 14 on a surface 16, such as a pillow (as shown). The device 10 is securable to a person's head 18 extending from the side of the head 30 in an area generally above the person's ear 20, across the forehead 22, to an area above the opposite ear (against the pillow in FIG. 1) to keep the face 14 elevated or angled away from the surface 16 while the person 12 is sleeping on his or her side. This configuration is believed to lift the temple area of the head 18, thereby lifting the face 14, particularly the cheek area, to reduce the pressure on it and thereby minimizing deepening wrinkles as a result of physical manipulation of the facial skin during sleep. It also can, over time, help repair or at least minimize deep set wrinkles that have appeared on the face.

With further reference to FIGS. 1, 2, 3, and 4, the article 10 of the present invention includes a longitudinally extending head support 24 having a first end 26 and a second end 28. The head support 24 is preferably flexible for conforming to the head 18, and is of sufficient length from end 26 to end 28 so as to extend from one side of the head 30 in an area generally and preferably above the person's ear 20, across the forehead 22, to the opposite side of the head (not shown) in an area generally and preferably above the person's other ear (not shown). It is appreciated that the area above the person's ear is an approximate position, and that this area includes the area generally above the ear and the person's temple area. In a more preferred embodiment, the head support 24 does not extend substantially beyond the ears to portions toward the rear of the person's head.

The head support 24 should be of sufficient thickness to elevate the temple area, and thereby elevate the face 14 relative to the surface/pillow 16 upon which it rests so as to minimize the pressure from the pillow 16 on the face 14 as further described below. A head securing means 32 for holding the head support 24 on the head, such as the elastic strap 34 shown, is attached to the first and second ends 26, 28 of the head support 24 and is preferably formed of first and second elastic sections 34a and 34b connectable to one another by Velcro connectors 36 or other suitable means as further described below.

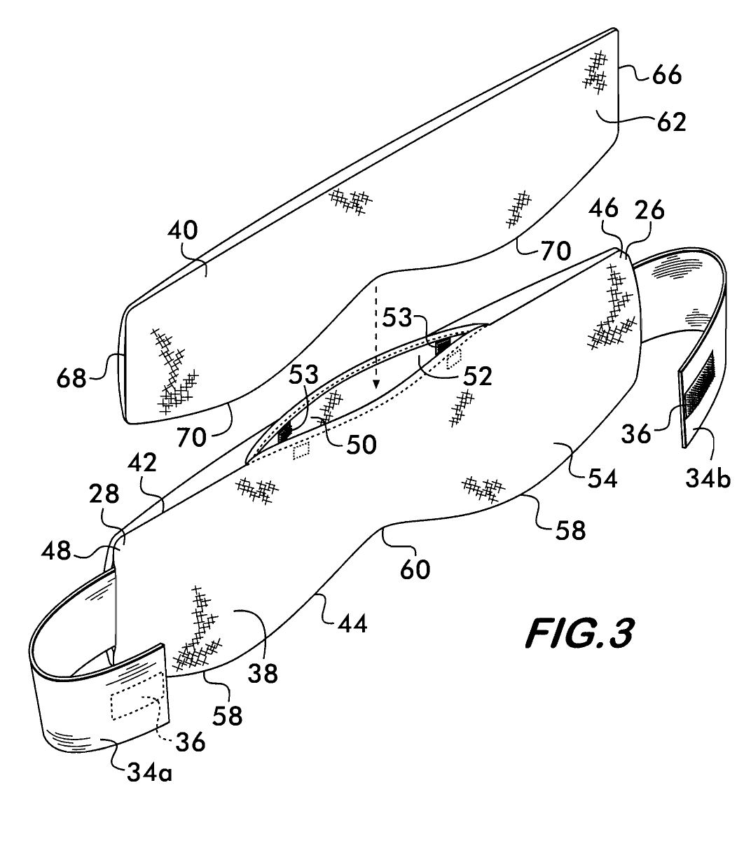

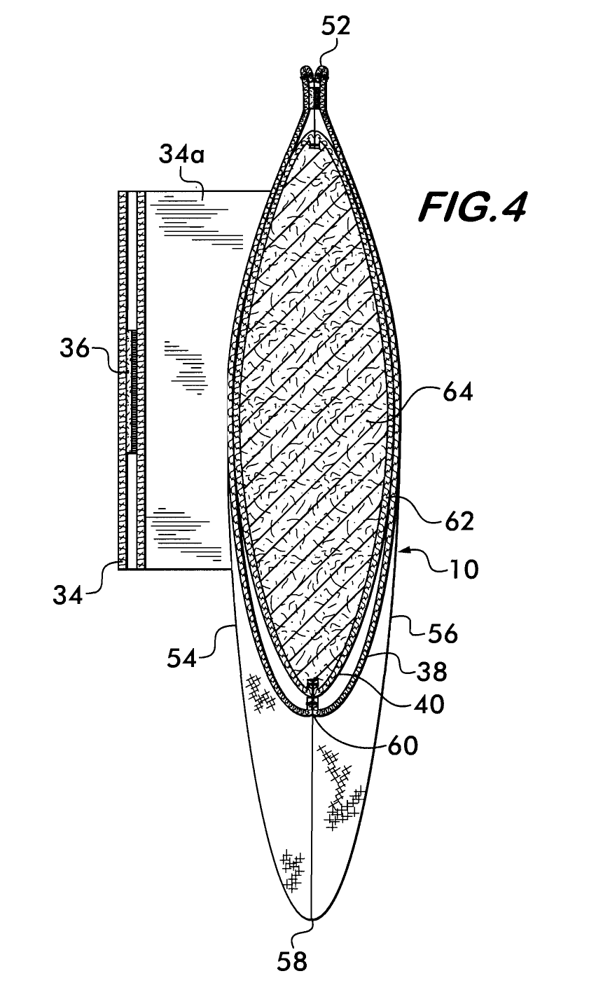

In the illustrated embodiment, the head support 24 includes a cover 38 and removable insert 40. Cover 38 is formed as a longitudinally extending shaped fabric member having a top 42, bottom 44, a first end 46 that defines the end 26 of the head support in the preferred embodiment, and a second end 48 that likewise defines the end 28 of the head support in the preferred embodiment. The cover 38 further forms a cavity 50 via any suitable means such as folding and stitching the fabric as is known in the art. A slit or opening 52 is formed along the top 42 of the cover 38 and is sized to easily receive the insert 40 into the cavity 50, as illustrated in FIG. 3. The slit 52 may include one or more securing members 53, such as Velcro, to maintain it in a closed position. Other suitable securing means may be used, such as snaps.

A first face 54 of the cover 38 rests against the skin or forehead of the person 12, while the second face 56 faces away from the person. In the preferred embodiment, the bottom 44 of the cover 38 may form a pair of rounded ends or extensions 58 separated by a nose recess 60, as shown, to allow the article 10 to be used optionally to cover the person's eyes in addition to providing wrinkle relief simply by lowering the head support 24 over the person's eyes.

The cover 38 may be made from any suitable material or combination of materials known in the art, particularly materials that are comfortable against the user's skin. The material is also preferably washable. In one embodiment, the cover 38 is made of cotton material. It may also be manufactured using multiple types of materials, such as a cotton with a woven non roll polyester or the like. In further embodiments, the material(s) may preferably be hypoallergenic.

In a preferred embodiment, the cover 38 is approximately dimensioned as follows: 400 mm in length from end to end (46, 48), 75 mm height at the ends (46, 48), 100 mm at the largest height of the two rounded bottom ends 58, and the slit 52 opening is 150 mm in length.

As previously discussed, the head securing means 32 is preferably formed as a strap 34 formed of first and second straps sections 34a and 34b, stitched to the ends 46 and 48 of the cover 38. The present invention, however, is not limited to such a securing means 32, and strap elements 34 may be secured together using alternative methods known in the art, such as, but not limited to, snaps, zipper, buckles, and the like. In further embodiments, the strap elements can simply be tied together. Any suitable securing means 32 may be used, including fabrics, laces, elastic materials, etc, and any suitable configuration may be used, such as the two straps sections or a single elastic member.

The insert 40 of the preferred embodiment is receivable within the cavity 50 through the slit 52. In this embodiment, the insert 40 conforms to and fills in the shape of the cover 38 so as to complete the head support 24 and provide the thickness of the head support for elevating the temple area. The insert also is preferably made of a resilient cushioning material to provide comfort to the user. While the insert 40 is illustrated as a single integral piece to extend longitudinally from the first end 46 to the second end 48 of the cover 38, the present invention is not so limited and may be comprised of multiple inserts fixed at designated points within the cover 38. For example, in one embodiment, a first insert may be adapted to fit within the first end 46 of the cover 38 such that the insert is substantially above the ear on the side of the person's head. A second insert may also be adapted to fit within the second end 48 of the cover such that the insert is substantially above the ear on the other side of the person's head. Finally, a third, and optional, insert may be secured within the forehead region of the cover 38 substantially between the first and second ends 46 and 48 of the cover 38. These three inserts may be separately provided in any combination or otherwise provided as a contiguous or continuous insert (as illustrated in FIG. 3) to form a cushioned area of the head support 24. In one embodiment, at least the first and second inserts are provided within the cover 38.

The insert 40 is preferably formed from any suitable material that is comfortable, resiliently compressible, and flexible to conform to the shape of the user's head. In the preferred embodiment, the insert 40 is formed of a removable elongated pillow like member having a fill material similar to that of a pillow. The outer layer 62 can be a cotton material or any other suitable material such as those used for the outer layer of a pillow. The fill material 64 within the outer layer 62 may include, cotton, polyester fiberfill, silicone, gels, combinations thereof, or the like. In further embodiments, the inserts and/or fill material may be hypoallergenic. The fill material 64 is preferably fully enclosed within the outer layer 62, e.g., the outer layer is stitched closed to prevent the fill material from escaping.

As previously noted, the head support 24 should be of sufficient thickness to lift the temple area of the user to minimize the pressure on the face 14, particularly the cheek area of the face, from the surface/pillow 16. In the preferred embodiment illustrated this is provided with an insert 40 having sufficient fill material 64 to provide the desired thickness. A head support having a thickness greater than about 1 centimeter is believed preferable, and at least approximately 2 centimeters in thickness more preferable, and a thickness of approximately 2.5 centimeters in thickness even more preferable. In the illustrated preferred embodiment, the insert is approximately 2.5 to 3 centimeters in thickness to provide the desired thickness for the head support 24 of about the same. In embodiments where a combination of inserts may be used at the first end 46, second end 48 and/or forehead regions 22, the insert thickness at each region may be uniform or have variable thickness. In the illustrated embodiment, the insert, in addition to being approximately 2.5 to 3 centimeters in thickness, is approximately 385 mm in length, 50 mm height at the ends 66, 68, and 75 mm at the largest height of the two rounded ends bottom 70.

The article 10 of the instant invention may also include an herbal or other similar scent to support aroma therapy for stress relief. Scents may be provided in any known manner such as scented sprays, beads, or other known additives and may be applied to or embedded within the cover or removable insert. In one embodiment, the scents are applied to or embedded within the fabric of the insert. In further embodiments, the scent may be applied or other wise included with the fill material 64.

As noted above and illustrated in FIG. 1, the article 10 of the present invention is secured to the head 18 of the person such that the head support 24 rests across his or her forehead, and the first end 26 and the second end 28 are at or above the general area of the ear of the subject's head. Again, this region includes the area of the side of a person's head above the ear including the temple region and preferably should not extend substantially beyond the ears to portions toward the rear of the person's head. The fastener elements (e.g. strap 34) should be adjusted such that the device fits snuggly and can be worn comfortably. When the person rests his or her head on a surface 16 (e.g. a pillow or mattress) the fill material 64 within the insert 46 provides a compressibly resilient comfortable support between at least the side of the person's head in the area of the temple and the surface 16, thus keeping the upper portion of the head elevated or angled. By angled, it is appreciated that if the thickness ends in the area above the ear, e.g., in the temple area, resting against the surface/pillow 16, this change in thickness from the head area resting directly on the surface 16 to the end of the head support 24 where the thickness begins, helps to slightly angle the head, and thus the face, away from the surface to minimize pressure on the face. Thus, the elevating affect maneuvers and/or redistributes the weight of the head such that the pressure exerted on the face by the surface is minimized. This is advantageous because it minimizes physical manipulation of the person's face by the surface to reduce and correct wrinkling.

Placement over or near the temples offers additional supporting counter pressure to this area, thus, providing an additional source of stress relief. In embodiments where the bulge 40 or other portion of the cover 38 are worn over the eyes, the instant invention provides the added benefit of blocking light while sleeping and can provide counter pressure to point at or about the eyes. In embodiments including an herbal or other similar scents, additional stress relief is provided.

While preferred embodiments have been described above, it is appreciated that other embodiments of the invention are contemplated. For example, the head support could be formed of a single integral member having similar properties as those of the cover 38 and insert 40, e.g., resilient, compressive, flexible, cushion like, and of suitable material to be comfortable against the skin, and of the preferred thickness. Other configurations are also possible. The article 10 also may provide additional advantages. For example, use of the article 10 may improve the quality of the person's sleep and make them feel more rested and refreshed in the morning.

What is claimed is:

1. A method of reducing facial wrinkles on a person when said person is resting one of its faces on a surface, such as a pillow, said method comprising:
securing an article to the person's head, said article having a cushioned section positioned substantially above a first face on a first side of said person's head in the area of the person's temple, said cushioned area having a thickness sufficient to elevate said first side of said head a sufficient distance to reduce the pressure on the first face from said surface when resting thereon; and
resting the first side of said person's head on the surface.

2. A method of reducing facial wrinkles in accordance with claim 1 wherein said method further includes securing said article to the person's head substantially above a second face on a second side of said person's head, said article having a second cushioned section positioned substantially above a second face on a second side of said person's head in the area of the person's other temple, said cushioned area having a thickness sufficient to reduce the pressure on the second face from said surface.

3. A method of reducing facial wrinkles in accordance with claim 2 wherein said article has a third cushioned area secured to the forehead of the person between said first and second cushioned areas above the nose of said person.

4. A method of reducing facial wrinkles in accordance with claim 1 wherein said first cushioned area is at least approximately 1 centimeter in thickness.

5. A method of reducing facial wrinkles in accordance with claim 1 wherein said first cushioned area is at least approximately 2 centimeter in thickness.

6. A method of reducing facial wrinkles in accordance with claim 1 wherein said first cushioned area is at least approximately 2.5 centimeter in thickness.

7. A method of reducing facial wrinkles in accordance with claim 1 wherein said first cushioned area has a thickness between approximately 2.5 to 3 centimeter in thickness.

8. A method of reducing facial wrinkles in accordance with claim 3 wherein said first, second and third cushioned areas form a contiguous cushioned area.

9. A method of reducing facial wrinkles in accordance with claim 1 wherein said cushioned area ends at said area approximately above the ear on said first side of said person's head.

10. An article worn on a person's head for reducing facial wrinkles when the person is resting one of its faces on a surface, comprising:
a longitudinally extending head support mountable on a person's forehead and having first and second ends, said head support being flexible for conforming to said forehead and of sufficient length between said first and second ends so as to be capable of extending from said first end positioned on the person's head at an area approximately above the person's ear, across the person's forehead, and to the second end positioned on an area approximately above the person's other ear, said head support being at least two centimeters in thickness prior to compression from use and cushioned at said first and second ends so as to cover at least the temple areas of the person; and
a strap connecting said first end to said second ends for extending around the back of the person's head to hold the article on the head.

11. An article in accordance with claim 10 wherein said head support comprises a cover and said an insert positioned within said cover, said strap being attached to said cover.

12. An article in accordance with claim 11 wherein the insert is received through an opening in the cover.

13. An article in accordance with claim 10 wherein said cushion material is formed of resiliently compressible material.

14. An article in accordance with claim 10 wherein said cushion material is comprised of a material selected from the group consisting of a cotton material, a woven non-roll polyester material, and combinations thereof, which is adapted to form a cavity filled with a material selected from the group consisting of polyester fiberfill, silicone, gels, and combinations thereof.

15. An article in accordance with claim 10 wherein said strap is formed of first and second sections attachable to one another via a securing member selected from the group consisting of one or more hook and loop assemblies, one or more snaps, one or more zippers, one or more buckles combinations thereof.

16. An article in accordance with claim 10 wherein the head support includes a top portion and a bottom portion where the bottom portion includes one or more bulge elements configured for covering the person's eyes.

17. An article in accordance with claim 10 wherein said head support includes a scented aroma embedded.

18. An article worn on a person's head for reducing facial wrinkles when the person is resting one of its faces on a surface, comprising:
a longitudinally extending head support mountable on a person's forehead and having first and second ends, said head support being cushioned and flexible for conforming to said forehead and of sufficient length between said first and second ends to be capable of extending from the person's approximate temple area on one side of the person's head, across the person's forehead, and to the person's approximate temple area on the other side of the person's head, said head support being at least about two centimeters in thickness prior to compression from use at least in the two temple areas; and
a strap connecting said first end to said second ends for extending around the back of the person's head to hold the article on the head.

19. The article for reducing facial wrinkles of claim 18 wherein said head support comprises a cover and an insert, said cover being formed of a flexible material and having a cavity for receiving said insert, said insert being cushioned and flexible for conforming to said forehead and containing a fill material to provide the at least about two centimeters in thickness.

20. The article for reducing facial wrinkles of claim 19 wherein said head support does not extend beyond the areas above the person's two ears.
